(19) United States
(12) Patent Application Publication
(10) Pub. No.: US 2017/0333441 A1
(43) Pub. Date: Nov. 23, 2017

(54) COMPOSITIONS FOR TREATING MICROBIAL INFECTIONS

(71) Applicant: Alcon Research, Ltd., Fort Worth, TX (US)

(72) Inventors: Malay Ghosh, Fort Worth, TX (US); Masood A. Chowhan, Arlington, TX (US)

(21) Appl. No.: 15/671,332

(22) Filed: Aug. 8, 2017

Related U.S. Application Data
(63) Continuation of application No. 14/632,547, filed on Feb. 26, 2015.
Application No. 14/632,547 is a continuation of application No. 13/152,051, filed on Jun. 2, 2011, now abandoned.
(60) Provisional application No. 61/350,820, filed on Jun. 2, 2010.

(51) Int. Cl. A61K 31/5383; A61K 33/30; A61K 33/06; A61K 45/06; A61K 9/00

(52) U.S. Cl. CPC A61K 31/5383; A61K 45/06; A61K 9/0048; A61K 33/06; A61K 33/30

(57) ABSTRACT

The present invention relates to methods for treating a microbial infection comprising treating the infected tissue with a composition comprising a quinolone such as finafloxacin and a divalent cation such as magnesium. The present invention also relates to antimicrobial compositions comprising a quinolone such as finafloxacin and a divalent cation such as magnesium.

### TECHNICAL FIELD OF THE INVENTION

The present invention generally relates to compositions for treating a microbial infection. The present invention specifically relates to methods for treating a microbial infection with a composition comprising a quinolone compound such as finafloxacin or a finafloxacin derivative and a divalent cation such as magnesium.

### BACKGROUND OF THE INVENTION

Microbial resistance to conventional antimicrobial treatment is an ongoing concern to medical professionals. Until the problem of resistance is overcome, a steady supply of new treatments and therapies for treating microbial infections is required in order to blunt the effect of microbe mutations that render conventional therapies less effective or, in certain cases, ineffective.

Quinolone compounds are known to have desirable antimicrobial properties. Certain quinolone compounds for use in the treatment of ophthalmic, otic, and nasal conditions are disclosed in U.S. Pat. No. 6,716,830, the entire contents of which are incorporated by reference herein.

A relatively new quinolone compound, finafloxacin, has been described as useful in the treatment of H. pylori infections. Buissonniere et al., “Antimicrobial activity of a new fluoroquinolone, finafloxacin, against H. Pylori in comparison to levofloxacin” Helicobacter, Vol. 13(5):465 (October 2008); U.S. Pat. No. 6,133,260 to Matzke et al. Ophthalmic, otic, and nasal applications of finafloxacin are not described in these references.

The use of compositions having antimicrobial properties is important for the treatment of infection (e.g., ophthalmic infections such as conjunctivitis). Infections such as ophthalmic conjunctivitis can be caused by various kinds of microbes, with most cases being due to bacteria and/or viruses. Unfortunately, conjunctivitis symptoms are not specific to the etiology of the infectious agent and significant testing may be required to determine the causative agent or microbe. Care must be taken in selecting appropriate agents for treating conjunctivitis, given the sensitive tissues affected by the infection. In view of the above-recited difficulties in treatment, compositions for treating conjunctivitis are needed that have broad-spectrum antimicrobial properties, a benign toxicological profile, and/or characteristics that prevent the transmission of contagious infectious agents.

Furthermore, certain quinolone compounds may have poor aqueous solubility, and various techniques have been used to improve quinolone solubility. Yamakawa et al., (J. Controlled Release Vol. 86:101-113, 2003) describes the solubilization of non-fluorinated quinolones using magnesium in combination with cyclodextrin and polyvinylpyrrolidine (PVP). However, fluoroquinolones such as finafloxacin were not tested, and the authors report that formulations without cyclodextrin and PVP were not stable during storage.

### BRIEF SUMMARY OF THE INVENTION

The present invention relates to compositions for the treatment of microbial infection comprising a quinolone antimicrobial compound such as finafloxacin or a pharmaceutically acceptable salt, derivative, enantiomer, or hydrate thereof. In preferred embodiments, such compositions are for the treatment of ophthalmic, otic, and nasal infections, particularly infections caused by various bacterial species. The compositions of the invention further comprise a divalent cation such as magnesium, zinc, or calcium to improve the aqueous solubility of the quinolone antimicrobial compound. Preferred quinolone compositions have an acidic pH and comprise magnesium as the divalent cation.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a graph showing the solubility of finafloxacin in three magnesium chloride-containing solutions as pH varies.

### DETAILED DESCRIPTION OF THE INVENTION

The compositions of the present invention comprise a quinolone antimicrobial in aqueous solution with a divalent cation. Preferred quinolone antimicrobials include those described in U.S. Pat. Nos. 4,990,517 and 5,059,597 to Petersen et al. (the entire contents of which are hereby incorporated by reference), and quinolones such as finafloxacin, ciprofloxacin, gatifloxacin, and ofloxacin. A particularly preferred quinolone is finafloxacin or a pharmaceutically acceptable salt, derivative, enantiomer, or hydrate thereof. Finafloxacin (8-cyano-1-cyclopropyl-6-fluoro-7-[(4aS, 7aS)-hexahydro pyrrolo[3,4-b]-1,4-oxazin-6(2H)-yl]-1,4-dihydro-4-oxo-3-quinoline carboxylic acid) has the following structure:

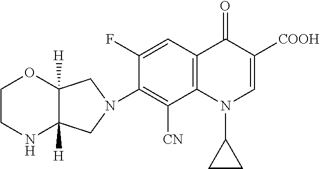 [SMILES: N#Cc1c(N2C[C@@H]3NCCO[C@H]3C2)c(F)cc2c(=O)c(C(=O)O)cn(C3CC3)c12]

A preferred quinolone salt for use in embodiments of the present invention is finafloxacin monohydrochloride. Diasteromerically and enantiomerically pure finafloxacin is also preferred for use in embodiments of the present invention. As used herein, the term finafloxacin is intended to encompass finafloxacin and its pharmaceutically acceptable salts, derivatives, enantiomers, or hydrates. The phrase “pharmaceutically acceptable” is art-recognized and refers to compositions, polymers and other materials and/or dosage forms which are suitable for use in contact with the tissues of human beings and animals without excessive toxicity, irritation, allergic response, or other problem or complication, commensurate with a reasonable benefit/risk ratio as determined by one of ordinary skill in the art.

Finafloxacin and derivatives thereof can be synthesized according to the methods described in U.S. Pat. No. 6,133,260 to Matzke et al., the contents of which are herein incorporated by reference in their entirety.

It is also contemplated that the concentrations of the ingredients comprising the compositions of the present invention can vary. In preferred compositions, a quinolone antimicrobial is present in ophthalmic or otic compositions at a concentration of about 0.1% to 1.0% w/v. In a particularly preferred embodiment, a quinolone antimicrobial is present at a concentration of about 0.1% to 0.5%. A person of ordinary skill in the art would understand that the concentrations can vary depending on the addition, substitution, and/or subtraction of ingredients in a given composition.

In addition to a quinolone compound, the compositions of the present invention additionally comprise a divalent cation such as magnesium, zinc, or calcium. In a preferred embodiment, magnesium is provided in the form of a pharmaceutically acceptable salt such as magnesium chloride. The divalent cation is generally present at a concentration of 1-100 mM, preferably at a concentration of 3-75 mM, and most preferably at a concentration of 4-50 mM.

Compositions of the present invention are generally prepared using a buffering system that maintains the composition at a pH of about 3 to a pH of about 8. In certain embodiments, topical compositions (particularly topical ophthalmic compositions, as noted above) are preferred which have a physiological pH matching the tissue to which the composition will be applied or dispensed. For certain quinolones such as finafloxacin, the compositions generally have an acidic pH of less than 7 and generally between 5 and 7. For otic applications, an acidic pH of between 5 and 6 is particularly preferred. A preferred buffering system uses sodium acetate at a concentration of 0.01 to 1.0 w/v % which may increase the solubility of finafloxacin in certain finafloxacin compositions of the present invention.

The compositions of the invention are particularly directed toward treating mammalian and human subjects having or at risk of having a microbial tissue infection. Microbial tissue infections that may be treated or prevented in accord with the method of the present invention are referred to in J. P. Sanford et al., “The Sanford Guide to Antimicrobial Therapy 2007” 37th Edition (Antimicrobial Therapy, Inc.). Particular microbial tissue infections that may be treatable by embodiments of the present invention include those infections caused by bacteria, protozoa, fungi, yeast, spores, and parasites. The present invention is also particularly directed to antimicrobial compositions for and methods of treating ophthalmic, otic, and nasal/sinus infections.

Certain embodiments of the present invention are particularly useful for treating ophthalmic tissue infections. Examples of ophthalmic conditions that may be treated using compositions and methods of the present invention include conjunctivitis, keratitis, blepharitis, dacyrocystitis, hordeolum and corneal ulcers. The methods and compositions of the invention may also be used prophylactically in various ophthalmic surgical procedures that create a risk of infection.

Otic and nasal/sinus tissue infections may also be treated by embodiments of the present invention. Examples of otic conditions that may be treated with compositions and methods of the present invention include otitis externa and otitis media, including those situations where the tympanic membrane has ruptured or tympanostomy tubes have been implanted. Examples of nasal/sinus conditions that may be treated with compositions and methods of the present invention include rhinitis, sinusitis, nasal carriage and situations where the nasal or sinus tissues are affected by surgery.

Embodiments of the present invention may also be used prophylactically to prevent infection of a tissue by an infectious agent. In such embodiments, a tissue at risk of infection is contacted with a composition of the present invention.

In particular embodiments, a composition of the present invention is administered once a day. However, the compositions of the present invention may also be formulated for administration at any frequency of administration, including once a week, once every 5 days, once every 3 days, once every 2 days, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. One of ordinary skill in the art would be familiar with determining a therapeutic regimen for a specific indication that incorporates a pharmaceutically effective amount of a composition of the present invention. The phrase “pharmaceutically effective amount” is an art-recognized term, and refers to an amount of an agent that, when incorporated into a pharmaceutical composition of the present invention, produces some desired effect at a reasonable benefit/risk ratio applicable to any medical treatment. The effective amount may vary depending on such factors as the disease or infectious agent being treated, the particular composition being administered, or the severity of the disease or infection agent.

In the methods set forth herein, administration to a subject of a pharmaceutically effective amount of a composition that includes a quinolone antimicrobial may be by any method known to those of ordinary skill in the art.

For example, the composition may be administered locally, topically, intradermally, intralesionally, intranasally, subcutaneously, orally, by inhalation, by injection, by localized perfusion bathing target cells directly, via a catheter, or via lavage.

In particular embodiments, the composition is administered topically to an ocular surface. Regarding ophthalmic administration, it is contemplated that all local routes to the eye may be used, including topical, subconjunctival, periocular, retrobulbar, subtenon, intraocular, subretinal, posterior juxtascleral, and suprachoroidal administration.

For ophthalmic administration, the composition may be a solution, a suspension, a gel, or an ointment. In preferred aspects, compositions will be formulated for topical application to the eye in aqueous solution in the form of drops. The term “aqueous” typically denotes an aqueous composition wherein the excipient is >50%, more preferably >75% and in particular >90% by weight water. These drops may be delivered from a single dose ampoule which may preferably be sterile and thus render bacteriostatic components of the composition unnecessary. Alternatively, the drops may be delivered from a multi-dose bottle.

In other aspects, components of the invention may be delivered to the eye as a concentrated gel or a similar vehicle, or as dissolvable inserts that are placed beneath the eyelids. In yet other aspects, components of the invention may be delivered to the eye as ointment, water-in-oil and oil-in-water emulsions.

For use in sinus and respiratory infection applications, compositions of the present invention may be administered via topical droplets or using nebulizers or other such devices that are suitable for aerosol delivery of the compositions.

Various otic administration techniques are also contemplated. In particular embodiments, a composition of the present invention may be delivered directly to the ear canal (for example: topical otic drops or ointments; slow release devices in the ear or implanted adjacent to the ear). Local administration routes include otic intramuscular, intratympanic cavity and intracochlear injection routes for the compositions. It is further contemplated that certain compositions of the invention may be formulated in intraotic inserts or implant devices. For instance, delivery of the compositions can be accomplished by endoscopic assisted (including laser-assisted endoscopy to make the incision into the tympanic membrane) injection into the tympanic cavity as set forth, for example, in Tsue et al., Amer. J. Otolaryngology, Vol. 16(3):158-164, 1995; Silverstein et al., Ear Nose Throat, Vol. 76:674-678, 1997; Silverstein et al., Otolaryngol Head Neck Surg, Vol. 120:649-655, 1999. Local administration can also be achieved by injection through the tympanic membrane using a fine (EMG recording) needle, through use of an indwelling catheter placed through a myringotomy incision, and injection or infusion through the Eustachian tube by means of a small tubal catheter. Furthermore, the compositions can be administered to the inner ear by placement of gelfoam (or similar absorbent and adherent product soaked with the compositions) against the window membrane of the middle/inner ear or adjacent structure with due discretion and caution by a skilled clinician. Various other devices can be used to deliver the compositions of the present invention to the affected ear compartment; for example, via catheter or as exemplified in U.S. Pat. No. 5,476,446 (incorporated by reference herein in its entirety) which provides a multi-functional apparatus specifically designed for use in treating and/or diagnosing the inner ear of the human subject. Also see U.S. Pat. No. 6,653,279 for other devices for this purpose (incorporated by reference herein in its entirety).

The compositions of the present invention optionally comprise one or more excipients. Excipients commonly used in pharmaceutical compositions include, but are not limited to, tonicity agents, preservatives, chelating agents, buffering agents, surfactants and antioxidants. Other excipients comprise solubilizing agents, stabilizing agents, comfort-enhancing agents, polymers, emollients, pH-adjusting agents and/or lubricants. Any of a variety of excipients may be used in compositions of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, xanthan gum, carrageenin, agar and acacia, starch derivatives, such as starch acetate and hydroxypropyl starch, and also other synthetic products such as polyvinyl alcohol, polyvinylpyrrolidone, polyvinyl methyl ether, polyethylene oxide, preferably cross-linked polyacrylic acid and mixtures of these products.

Suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, ionic and nonionic surfactants, though nonionic surfactants are preferred, RLM 100, POE 20 cetylstearyl ethers such as Procol® CS20 and poloxamers such as Pluronic® F68. Suitable antioxidants include, but are not limited to, sulfites, ascorbates, butylated hydroxyanisole (BHA) and butylated hydroxytoluene (BHT).

The compositions set forth herein may comprise one or more preservatives. Examples of such preservatives include p-hydroxybenzoic acid ester, alkyl-mercury salts of thiosalicylic acid, such as thiomersal, phenylmercuric nitrate, phenylmercuric acetate, phenylmercuric borate, sodium perborate, sodium chlorite, benzalkonium chloride, parabens such as methylparaben or propylparaben, alcohols such as chlorobutanol, benzyl alcohol or phenyl ethanol, guanidine derivatives such as polyhexamethylene biguanide, sodium perborate, or sorbic acid. In certain embodiments, the composition may be self-preserved that no preservation agent is required.

In certain embodiments, the compositions of the present invention include one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; vinyl polymers, such as polyvinyl alcohol; and carbomers, such as carbomer 934P, carbomer 941, carbomer 940 and carbomer 974P. Certain compositions of the present invention may be used with contact lenses or other ophthalmic products.

For topical ocular applications, the compositions are preferably isotonic, or slightly hypotonic in order to combat any hypertonicity of tears caused by evaporation and/or disease. This may require a tonicity agent to bring the osmolality of the composition to a level at or near 210-320 milliosmoles per kilogram (mOsm/kg). The pH of the solution may be in an ophthalmic acceptable range of 3.0 to 8.0. The ophthalmic compositions of the present invention generally have an osmolality in the range of 220-320 mOsm/kg, and preferably have an osmolality in the range of 235-300 mOsm/kg. The ophthalmic compositions will generally be formulated as sterile aqueous solutions.

In certain embodiments, the compositions set forth herein have a viscosity of 0.5-100 cps, preferably 0.5-50 cps, and most preferably 1-20 cps. This relatively low viscosity insures that the product is comfortable, does not cause blurring (in ophthalmic compositions), and is easily processed during manufacturing, transfer and filling operations.

The compositions of the present invention may also comprise an anti-inflammatory agent. The compositions of the present invention may also contain one or more anti-inflammatory agents. The anti-inflammatory agents utilized in the present invention are broadly classified as steroidal or non-steroidal. The preferred steroidal anti-inflammatory agents are glucocorticoids. Glucocorticoids for ophthalmic, otic, or nasal use include dexamethasone, loteprednol, rimexolone, prednisolone, fluorometholone, hydrocortisone, mometasone, fluticasone, beclomethasone, flunisolide, triamcinolone and budesonide. Non-steroidal anti-inflammatory agents are: prostaglandin H synthetase inhibitors (Cox I or Cox II), also referred to as cyclooxygenase type I and type II inhibitors, such as diclofenac, flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam and carprofen; cyclooxygenase type II selective inhibitors, such as NS-398, vioxx, celecoxib, P54, etodolac, L-804600 and S-33516; PAF antagonists, such as SR-27417, A-137491, ABT-299, apafant, bepafant, minopafant, E-6123, BN-50727, nupafant and modipafant; PDE IV inhibitors, such as ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast; inhibitors of cytokine production, such as inhibitors of the NF.kappa.B transcription factor, or other anti-inflammatory agents known to those skilled in the art. Anti-inflammatory agents included in the compositions of the present invention are generally at a concentration of about 0.01 to about 1.0 w/v %.

The following examples are presented to further illustrate selected embodiments of the present invention.

### Example 1

| Ingredient | % w/v |
|---|---|
| Finafloxacin | 0.33 |
| Magnesium Chloride (hexahydrate) | 0.3 |
| Sodium Acetate (trihydrate) | 0.68 |
| Mannitol | 2.5 |
| Benzalkonium Chloride | 0.01 |
| Sodium Hydroxide/Hydrochloric Acid | q.s. to pH 5.9 |
| Purified Water | q.s. 100% |

### Example 2

| Ingredient | % w/v |
|---|---|
| Quinolone antimicrobial | 0.10 to 1.0 |
| Zinc chloride | 0.3-1.0 |
| Sodium phosphate (anhydrous) | 0.3-0.7 |
| Sodium chloride | 0.7* |
| Sodium hydroxide/HCL | Adjust pH to 5.5 to 7.5 |
| Purified Water | q.s. 100% |

### Example 3—Solubility Studies

TABLE 1 below summarizes the results of solubility studies using finafloxacin monohydrochloride in aqueous solution with various salts at different concentrations. As shown in TABLE 1, magnesium, zinc, and calcium salts generated clear colorless solutions (denoted by C.C.S. in TABLE 1) at various pH ranges. In particular, magnesium salts generated clear, colorless solutions at slightly acidic, physiologically-compatible pH ranges.

TABLE 1

| | Concentration | | Conc. (%) | C.C.S. | |
|---|---|---|---|---|---|
| Salt | of salt (mM) | | Finafloxacin | pH Range | Observation |
| NaCl | 120 | mM | 0.79% | Not Formed | Suspension at pH 3.7-7.4 |
| | 27 | mM | 0.15% | Not Formed | Suspension at pH 3.7-7.4 |
| | 13.5 | mM | 0.076% | Not Formed | Suspension at pH 3.7-7.4 |
| NaSO₄ | 21.3 | mM | 0.3% | Not Formed | Suspension at pH 3.7-7.4 |
| KCl | 93 | mM | 0.7% | Not Formed | Suspension at pH 3.7-7.4 |
| AlCl₃ | 13.5 | mM | 0.180% | 2.50-7.24 | Pale-yellow → at pH 7.41 |
| | | | | | sample was clear but after |
| | | | | | 15 minutes suspension was |
| | | | | | formed |
| | 6.7 | mM | 0.09% | 2.83-7.46 | Pale-yellow→at pH 7.58 |
| | | | | | suspension was formed |
| | 1.3 | mM | 0.018% | Not Formed | Suspension at pH 3.7-7.4 |
| MgCl₂ | 13.5 | mM | 0.12% | 5.26-9.61 | C.C.S |
| | 6.7 | mM | 0.064% | 6.00-9.84 | C.C.S |
| | 4.91 | mM | 0.046% | 6.68-7.65 | C.C.S |
| | 2.45 | mM | 0.023% | Not Formed | Suspension at pH 3.7-7.4 |
| | 1.3 | mM | 0.012% | Not Formed | Suspension at pH 3.7-7.4 |
| Mg(CH₃COO)₂ | 13.5 | mM | 0.19% | 5.21-8.51 | C.C.S. |
| | 6.7 | mM | 0.095% | 6.05-7.57 | C.C.S. |
| | 1.3 | mM | 0.019% | Not Formed | Suspension at pH 3.7-7.4 |
| MgSO₄ | 13.5 | mM | 0.16% | 6.29-7.44 | C.C.S |
| | 6.7 | mM | 0.08% | 6.36-9.23 | C.C.S |
| | 1.3 | mM | 0.016% | Not Formed | Suspension at pH 3.7-7.4 |
| ZnCl₂ | 20.0 | mM | 0.27% | 5.06-5.87 | C.C.S |
| | 13.5 | mM | 0.184% | 4.50-5.55 | C.C.S |
| | 6.7 | mM | 0.09% | 4.90-5.47 | C.C.S |
| | 1.3 | mM | 0.018% | Not Formed | Suspension at pH 3.7-7.4 |
| ZnSO₄ | 13.5 | mM | 0.22% | 5.65 | C.C.S |
| CaCl₂ | 20.0 | mM | 0.22% | 6.29-6.39 | C.C.S |
| | 13.5 | mM | 0.15% | 6.45 | C.C.S |
| | 6.7 | mM | 0.075% | Not Formed | Suspension at pH 3.7 |
| | 1.3 | mM | 0.015% | Not Formed | Suspension at pH 3.7 |
| CaGluconate | 17.5 | mM | 0.75% | Not Formed | Suspension at pH 3.7 |
| CuCl₂ | 13.5 | mM | 0.18% | 5.77-6.11 | Light blue coloration |
| | 6.7 | mM | 0.09% | 5.14-7.26 | Light blue coloration |
| | 1.3 | mM | 0.018% | Not Formed | Suspension at pH 3.7-7.4 |
| 0.1% MgCl₂ | 10.5 | mM | 0.1% | 4.68-5.88 | C.C.S |
| 0.1% ZnCl₂ | 7.3 | mM | 0.1% | | |
| 0.1% MgCl₂ | 10.5 | mM | 0.1% | 5.01-5.75 | C.C.S |
| 0.05% ZnCl₂ | 3.67 | mM | 0.05% | | |
| 0.05% MgCl₂ | 5.27 | mM | 0.05% | 4.62-5.86 | C.C.S |
| 0.1% ZnCl₂ | 7.34 | mM | 0.1% | | |
| 0.05% MgCl₂ | 5.27 | mM | 0.05% | 4.86-5.87 | C.C.S |
| 0.05% ZnCl₂ | 3.67 | mM | 0.05% | | |

TABLE 2 below presents the results of solubility studies involving the fluoroquinolone antimicrobials ciprofloxacin, ofloxacin, gatifloxacin. The literature-reported aqueous solubilities for these compounds at pH 7.4 in 0.1M phosphate buffer are 0.02 w/v %, 0.35 w/v %, and 0.21 w/v %, respectively. Ciprofloxacin initially forms a solution in water and then precipitates out to form a suspension after reaching a pH of 4.69, 5.30 and 5.76 for the ZnCl₂, MgCl₂, and CaCl₂ solutions described in TABLE 2 below. Ofloxacin and gatifloxacin initially form a suspension in water. However, after the addition of ZnCl₂ or MgCl₂ these compounds become soluble. After the addition of CaCl₂, these compounds form less turbid solutions, with some particulate matter remaining in suspension. In summary, aqueous solubilities of the quinolone antimicrobials tested increased when the quinolone formulations included a divalent cation such as magnesium, zinc, or calcium.

TABLE 2

| | | | | |
|---|---|---|---|---|
| | Ion | Quinolone | | |
| Ion | Concentration | Conc. | C.C.S. | |
| Source | mM | (w/v %) | pH Range | Observation |

CIPROFLOXACIN (0.3%)

| | | | | |
|---|---|---|---|---|
| ZnCl₂ | 10.0 mM | 0.13% | 3.46-4.69 | C.C.S |
| MgCl₂ | 10.0 mM | 0.09% | 4.03-5.30 | C.C.S |
| CaCl₂ | 10.0 mM | 0.08% | 4.60-5.76 | C.C.S |

OFLOXACIN (0.3%)

| | | | | |
|---|---|---|---|---|
| ZnCl₂ | 10.0 mM | 0.13% | 6.20-7.24 | C.C.S |
| MgCl₂ | 10.0 mM | 0.09% | 6.54-7.30 | C.C.S |
| CaCl₂ | 10.0 mM | 0.08% | 7.42-7.53 | C.C.S |

GATIFLOXACIN (0.3%)

| | | | | |
|---|---|---|---|---|
| ZnCl₂ | 10.0 mM | 0.13% | 6.61-7.31 | C.C.S |
| MgCl₂ | 10.0 mM | 0.09% | 6.97-7.52 | C.C.S |
| CaCl₂ | 10.0 mM | 0.08% | 7.31-7.55 | C.C.S |

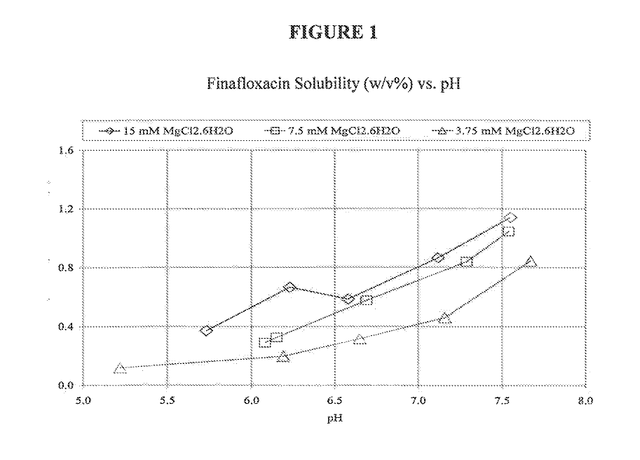

TABLES 3A, 3B, and 3C below show the solubility of finafloxacin in three magnesium chloride-containing finafloxacin solutions at various pH levels. Additional finafloxacin data demonstrating solubility at various pH levels is shown in FIG. 1.

TABLE 3A

| Final | Finafloxacin | Finafloxacin |
|---|---|---|
| pH | Free-Zwitterion, w/v % | HCl, w/v % |
| 5.73 | 0.370 | 0.404 |
| 6.23 | 0.666 | 0.727 |
| 6.58 | 0.585 | 0.638 |
| 7.12 | 0.865 | 0.944 |
| 7.55 | 1.140 | 1.244 |

TABLE 3B

| | Finafloxacin | Finafloxacin |
|---|---|---|
| pH | Free-Zwitterion, w/v % | HCl, w/v % |
| 6.08 | 0.290 | 0.316 |
| 6.15 | 0.325 | 0.355 |
| 6.69 | 0.576 | 0.628 |
| 7.29 | 0.839 | 0.915 |
| 7.54 | 1.045 | 1.140 |

TABLE 3C

| | Finafloxacin | Finafloxacin |
|---|---|---|
| pH | Free-Zwitterion, w/v % | HCl, w/v % |
| 5.22 | 0.119 | 0.130 |
| 6.19 | 0.198 | 0.216 |
| 6.65 | 0.317 | 0.346 |
| 7.16 | 0.459 | 0.501 |
| 7.67 | 0.844 | 0.921 |

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. A pharmaceutically acceptable composition comprising a quinolone antimicrobial or a pharmaceutically acceptable salt thereof and more than one divalent cation, said composition suitable for the treatment of an ophthalmic infection by topical ocular administration, and wherein said quinolone antimicrobial is finafloxacin at a concentration of 0.1 to 1.0 w/v %, said divalent cations are magnesium at a concentration of 1 to 100 mM, and zinc at a concentration of 1 to 100 mM, and said composition has a pH of 4.5 to 7.5, said composition further comprising sodium acetate at a concentration of 0.01 to 1.0 w/v %.

2. A composition according to claim 1 wherein the disorder is an ophthalmic, otic, or nasal infection.

3. A composition according to claim 1, further comprising an anti-inflammatory agent.

4. A composition according to claim 1, comprising a quinolone antimicrobial or a pharmaceutically acceptable salt thereof at a concentration of 0.1 to 0.5 w/v %.

5. A composition according to claim 1 wherein said divalent cations are each present at a concentration of 3 to 75 mM.

6. A composition according to claim 1 wherein said divalent cation are each present at a concentration of 4 to 50 mM.

7. A composition according to claim 1, said composition having a pH of 5.0 to 6.0.

8. A composition according to claim 1 wherein said magnesium divalent cation is present at a concentration of about 10 mM and said zinc divalent cation is present at a concentration of about 7.3 mM.
